(19) United States
(12) Patent Application Publication
(10) Pub. No.: US 2012/0130327 A1
(43) Pub. Date: May 24, 2012

(54) PERFECTING OF THE IMPROVED VACUUM DRESSING AND THE USE THEREOF IN VACUUM-ASSISTED THERAPY

(76) Inventor: Juan Marquez Cañada, Valencia (ES)

(21) Appl. No.: 13/364,164

(22) Filed: Feb. 1, 2012

Related U.S. Application Data
(63) Continuation-in-part of application No. 13/132,435, filed on Jun. 2, 2011.

(30) Foreign Application Priority Data
May 20, 2009 (ES) U200900931

(51) Int. Cl. A61M 1/00

(52) U.S. Cl. 604/319

(57) ABSTRACT

The perfecting of the improved vacuum dressing and the use thereof in vacuum-assisted therapy may be a tool that may include the possibility of incorporating a drainage device with modified Redon drains and may increase or reduce the variable permeability of said dressing in a manufactured manner, depending on the designation thereof.

### CROSS-REFERENCE TO RELATED APPLICATIONS

This application is claims priority to and benefit of the following applications: (1) U.S. patent application Ser. No. 13/132,435, filed on Jun. 2, 2011; (2) Patent Cooperation Treaty Application PCT/ES2010/000221, filed on May 20, 2010; and (3) Spain Patent Application No. 0200900931, filed on May 20, 2009. Each of the above cited patents is hereby incorporated by reference as if fully set forth in its entirety.

### DESCRIPTION

### Object of the Invention

The object of this application is the perfecting of the improved vacuum dressing (hereinafter, IVD) of the PCT2010000221, of the same applicant; said PCT, focuses on a vacuum dressing, of the type that incorporates, beneath its sealing film, a core with an outer open-pore component and an inner component which has a permeability that is variable, whose structural design makes it suitable to promote healing in losses of substance, but also as a tool in those situations that require compression; as it can be applied not only on the bed of the wound or loss of substance, but also directly on the cutaneous surface and, potentially, on various noble tissues or structures of said wounds or losses of substance, unlike the conventional vacuum dressings currently used. Moreover, it includes the possibility of incorporating a drainage device with modified Redon drains.

The present invention relates to the perfecting of said IVD; which has been perfected either to increase the flexibility or versatility of application of said dressing (possibility of incorporating a 2^{nd} inner component and, moreover, even said 2^{nd} inner component have its own drainage device; possibility of being manufactured with different geometrical configurations specific for the shapes of the surfaces of contact, possibility to be manufactured with oblique cuts in several directions to “universal” adaptation to different locations; possibility that the union of its inner and outer components could be reversible for added versatility, etc) or to expand even more the indications of vacuum-assisted therapy (hereinafter, VAT) by increasing or reducing the variable permeability of said dressing in a manufactured manner, depending on the designation thereof.

### FIELD OF APPLICATION OF THE INVENTION

The field of application of the present invention falls within the technical sector of the industry dedicated to the manufacturing of sanitary medical material, particularly dressings, bandages and similar devices for wounds and/or surgical planes.

### BACKGROUND OF THE INVENTION

As regards the Pathophysiology of Wounds, it is important to bear in mind that one of the main foundations for a wound to evolve favourably is combating the oedema.

In general, there are two different types of wounds or lesions:

1. Closed lesions or wounds. In them, the oedema is produced by the causal agent itself (blow, fall, etc., or even the aggression involved in the surgical procedure).
2. Open lesions or wounds. In them, the oedema is caused by a double aggression:
An initial aggression, already mentioned, produced by the causal agent itself.
An additional aggression, arising from direct exposure or contact of the open wound bed with air, the environment or, even, in the event that there is associated necrosis, the necrotic tissue itself.

Therefore, basically, in order to combat the oedema:

1. In closed lesions or wounds, the anti-oedema therapy will primarily consist of compression therapy (i.e. positive pressure), with or without the inclusion of drainages.
2. In open lesions or wounds, the first measure to combat the oedema, in the absence of necrosis, will be to protect them from the exterior by means of dressings which contribute to create a favourable, non-hostile environment that promotes tissue regeneration by favouring the elimination of the lesion exudate, as well as the formation of granulation tissue and a correct epithelialisation; whereas, in the presence of necrosis, the actions, in addition to protection of the wound from the exterior, shall be aimed at debridement or elimination of the detritus material found in the wound bed. In both cases, we should bear in mind that the healing effect may be increased by using associated compression, because this will enhance the anti-oedema effect.

Thus, in general, 3 stages may be considered in the healing process of an open wound:

Stage I. Debridement.

Stage II. Granulation.

Stage III. Epithelialisation.

If we analyse the methods used for the treatment of wounds through time, we find that, from the second half of the 20th century, there was a great advance in the healing of wounds, with the appearance and development of different types of dressings, called new-generation atraumatic or protective dressings, which attempt to mimic the characteristics of human skin in order to allow for a more effective insulation of the wound, maintaining a physiological environment, whereas the inner surface thereof (designed to come in contact with both the wound and the perilesional skin) “protects” the wound and the perilesional skin; these “atraumatic or protective” dressings make it possible to absorb the exudate, but prevent macro-penetration of the granulation tissue neo-formed in the interior thereof; consequently, their removal from the wound is also “atraumatic” and, therefore, they protect epithelialisation. Moreover, in the event that there is associated necrosis, they can facilitate the debridement or elimination of the detritus material found in the wound bed. However, despite their advantages, they continue to have a limited absorption capacity (such that, depending on the exudate, sometimes they may remain in situ for more than 7 days and, on the contrary, sometimes they require more frequent changes) and, furthermore, they continue to require supplementary compression bandages, which, despite the advances mentioned in regards to the healing of wounds, have not undergone significant changes, since they continue to be circular.

In the last decade of the 20th century, thanks to the company KCI (Kinetic Concepts Inc., San Antonio, Tex.), a new concept in the treatment of wounds emerged, with “Reduced or Negative Pressure Therapy”. The system introduced by KCI showed that application of a vacuum pressure on wound beds experimentally and clinically accelerated tissue repair processes.

Thus, one of the currently most widely used devices in vacuum-assisted wound therapy is the “VAC®” (Vacuum-Assisted Closure) system, manufactured by KCI. This system consists of placing a sterile cross-linked polyurethane (PU) foam dressing with open pores (400-600 μm) over the entire surface of the wound. An aspiration tube connected to a programmable vacuum pump is applied on this sponge. The vacuum pump is endowed with a reservoir to store the fluid extracted from the wound. The sponge and the tube outlet are sealed and insulated from the exterior by means of an adhesive film that adheres to the surrounding skin, creating a closed system. The high porosity of the polyurethane foam dressing used in this system, derived from its regular open-macropore structure, causes the pressure to be uniformly distributed throughout the entire surface of the wound; for this, it is essential to ensure that the system is hermetic and that there are no leakages through the insulating plastic dressing. As a result, this dressing with big open pores allows for direct transmission of the Reduced or Negative Pressure (vacuum) generated in a suction source to the wound; in theory, this confers it with an unlimited absorption capacity (unlike previous “atraumatic” dressings).

According to the manufacturer, the mechanisms whereby the application of Reduced Pressure, lower than atmospheric pressure or the suction pressure, on a wound makes it possible to reduce the oedema thereof and promote tissue repair processes would be macrostress (at the tissue level) and microstress (at the cellular level), which would act jointly. These mechanisms would be caused by the application of the reduction in pressure and direct contact between the open-pore foam and the wound bed.

However, this hypothesis to explain the anti-oedema effect of Reduced Pressure Therapy does not consider the fact that the basic mechanism for oedema reduction is compression (i.e. positive pressure, greater than atmospheric pressure). Moreover, these Conventional Vacuum Dressings (hereinafter, CVDs) exhibite a number of disadvantages that prevent them from coming in contact with the noble parts of wounds (tendons, bone, visceral tissue, etc.) or the perilesional skin, and from being able to keep it “in situ” for more than 2-3 days. Therefore, they entailed a “loss of opportunity”, since the system's unlimited absorption capacity did not necessarily translate into a smaller number of dressing changes as compared to “atraumatic or protective” dressings; moreover, they require more time to be put in place (to be cut according to the shape of the wound).

In fact, what clinical experience has always shown is that, upon removing the CVD in each dressing change, a trace remained on the surface of the body beneath it. This meant not only that, beneath the CVD, the system also generated positive pressures, greater than atmospheric pressure, but, moreover, that the final result of Reduced Pressure Therapy was Positive, because, although it is true that the application of vacuum therapy on the surface of the body generates reduced pressures at the level of the dressing pores, it is also true that, at the level of the points of contact between the solid components of the foam (pore walls) and the wound bed, the system generates positive pressures.

Thus, a VAT dressing was needed that would make it possible to expand the indications of this Vacuum Therapy, not only to promote healing in losses of substance, but also as a tool in those situations that require compression, whether or not there is a loss of substance, such as:
Lipoaspiration in areas that are difficult to compress with conventional pressotherapy garments/belts (for example, cervical-dorsal lipomatosis, localised lipomatosis in morbid obesity, etc.).
Surgical procedures in general (for example, abdominoplasties), in order to minimise the risk of post-operative morbidity, since VAT, as compared to conventional belts/bandages, would represent an “active” compression system, which, amongst others, would combine the properties of being aspirational, selective, controlled and centripetal; it would also offer the possibility of incorporating a system of Redon drains integrated therein; consequently, in addition to reducing stress on the suture lines, it would make it possible to dispense with conventional bandages and external Redon drains; this would make it possible to reduce the bed confinement period and facilitate the patient's precocious mobilisation.
Prevention/treatment of pathological scars
Treatment of oedemas of non-surgical origin
Stabilisation or splinting following sprains or luxations

However, the significance of the Negative or Positive sign of the pressure is essential not only to better understand the mechanism of action of the so-called “Reduced or Negative Pressure Therapy” and, therefore, to be able to expand the applications thereof, but also to prevent its adverse effects.

Along the same lines, currently there are publications about the “paradox” inherent in the fact that the final result of Reduced or Negative Pressure Therapy is Positive, which also warn about the risks that this may entail:
Negative-Pressure Wound Therapy I: The Paradox of Negative-Pressure Wound Therapy (Plast. Reconstr. Surg. 123: 589-598, 2009).
Negative-Pressure Wound Therapy II: Negative-Pressure Wound Therapy and Increased Perfusion. Just an Illusion? (Plast. Reconstr. Surg. 123: 601-612, 2009).
The Paradox of Negative-Pressure Wound Therapy—In Vitro Studies (JPRAS. 63: 174-179, 2010).

Moreover, the fact that the final result is Positive means that, in addition to a loss of time because it has to be cut (in order to prevent contact with the perilesional skin), the CVD also entails a loss of opportunity and effectiveness, since, given that it cannot also be applied on the perilesional skin, it is not possible to optimise reduction of the perilesional oedema (present in all wounds or lesions) or approximation of the edges of the wound.

There are other systems that, instead of the PU foam, simply use gauze, but they have a lower effect on contraction of the lesion bed and, furthermore, they also cannot come in contact with the perilesional skin.

Therefore, such dressings with open macropores have been conceived/designed to be applied only on losses of substance/ulcers and should not come in contact with healthy skin. In fact, the most frequent complication caused by these systems is irritation, maceration and even ulceration of the skin, or also of the noble structures of the wound that may accidentally come in contact with the foam. Consequently, it would be necessary to place a type of atraumatic dressing, with a variable permeability that protects healthy skin, between these dressings and the surface of the body. There are numerous dressings in the market that may serve as a basis to meet these conditions (Tielle®, Mepilex®, Biatain®, Epifoam®, Skinfoam®, Allevyn®, Therafoam®, etc.).

These dressings are generally composed of three layers:
An outer layer, generally made of polyurethane that is permeable to steam and impermeable to fluids.
An intermediate, central hydropolymer layer with a high absorption capacity (generally made of polyurethane foam).
A micro-perforated inner layer (adhesive or not) generally made of polyurethane or silicone.

However, since they were not conceived as a vacuum-assisted compression treatment, such dressings, as in the case of the CVD foam, have the disadvantage that the available dimensions thereof, especially in the case of large lesion areas, do not make them suitable for this purpose and, although it is possible to couple them to one another when used as a part of an “aspiration” compression system using vacuum, which, in addition to aspirating the exudate, is selective, controlled and centripetal, the process is not very effective, it is tedious and requires dedicating additional time. Moreover, the risks of overlap or shearing between them prevent them from being kept in situ for more than 48 hours, in order to prevent potential adverse effects.

The IVD of the PCT2010000221 and the VAT

Therefore, the objective of the PCT2010000221 was to create an integrated dressing which:
made it possible to expand the indications of VAT as it was suitable to come in contact with healthy skin, and also, potentially, with various noble tissues or structures of said wounds or losses of substance, either as a primary or secondary dressing.
had also suitable dimensions for larger areas of the body.
had sufficient thickness to optimise compression.
allowed for the incorporation of a drainage device with modified tubes or Redon drains, in order to directly transmit the reduced pressures from the underlying bleeding area of the wound to the open-pore component of the dressing.

Moreover, the fact that it is also an integrated dressing suitable for being in direct contact with the skin would increase the effectiveness thereof, since it would make it possible to develop tangential centripetal pressures on the cutaneous surface, not only on the periphery of the dressing (at the level of the outer adhesive film that adheres it to the surrounding skin), but also directly beneath the surface of said dressing, thereby increasing cutaneous retraction and reduction of stress on the suture lines or contraction of the edges of the lesion bed; this will be enhanced even further in the case of dressings wherein said contact surface of the dressing with the skin is also adhesive. This is not possible with CVDs, since these cannot come in direct contact with the cutaneous surface.

Thus, the IVD has developed part of the vast potentiality provided by VAT, in the treatment of open or closed lesions or wounds. The applicant considers that the term “Vacuum-Assisted Therapy” would induce less error than “Negative-Pressure Therapy”, which, however, is more widely used. Thus, as this IVD has allowed VAT to be also applied on the perilesional skin, it has also made it possible to provide a selective, controlled, centripetal compression, enhanced by the conceptual advance of this “unlimited” absorption therapy system, which, instead of accumulating the exudate in the dressing, directs it towards a reservoir located in an external device.

Specifically, the IVD has focused on a dressing, applicable to surgical planes or lesion areas which required anti-oedema therapy; this dressing comprises, beneath its sealing film, which adheres to the skin on the periphery of said dressing, a core composed of an open-pore outer area or outer component (hereinafter, OC), as a retraction vector, since the open-pore structure makes it possible to transmit the vacuum in the interior thereof; and an inner component (hereinafter, IC), with a variable permeability, as a protection factor for the surface of the body, which will also retract upon being “dragged” by said OC; this component will be the one to come in direct contact with the surface of the body, thereby preventing the OC from doing so; said IC may be adhesive or not, and on said sealing film that covers said core there is an aspiration tube connected to a vacuum pump, the junction between said tube and the dressing being hermetically sealed.

Moreover, the greater thickness of the open-pore OC enhances positive sagittal pressures, thereby providing a greater compression effect and, moreover, allowing for the additional incorporation of a drainage device composed of modified evacuation tubes or Redon drains at the ends whereof multiple orifices have been made (in a manufactured manner), which directly transmit the reduced pressures from the surgical plane or bleeding lesion area to the open-pore area, thereby promoting tissue adhesion and avoiding the need to use external tubes or Redon drains.

Advantages of the IVD

Likewise, it is worth noting that the fact that the IVD is an integrated dressing which may also be applied on intact cutaneous surfaces will increase the effectiveness thereof, since it will make it possible to develop tangential centripetal pressures on the intact cutaneous surface, not only in the periphery of the dressing (at the level of the outer adhesive film that adheres it to the surrounding skin), but also directly beneath the inner surface of the core of said dressing, thereby providing greater cutaneous retraction and reduction of stress on the suture lines or contraction of the lesion bed. This is not possible with CVDs, since these may not come in direct contact with the cutaneous surface. And it will be enhanced even further in the case of IVDs with cores the inner surface whereof is also adhesive.

Another advantage of the IVD is that its permeability is easily “customisable” or adaptable “in situ” to the needs, as a function of the type of lesion. Thus, if it is applied on potentially or highly exudative lesions, it will be sufficient to pierce the IC with a sterile needle (or with the blade of a scalpel, if a more permeable dressing is required) in the desired areas (e.g. in the case of open wounds, it will generally be at the central area thereof) in order to increase its permeability; on the contrary, if it is applied on closed wounds, in general it will not be necessary to do so (although it may be done in accordance with the incision trajectory).

Another advantage or possibility of the IVD in clinical practise is that it might potentially behave like a dressing with “self-regulatory” permeability when a suction source is applied, such that, if the wound is very exudative, the micropores of the semipermeable membrane of the IC might dilate without the need to make perforations or punctures in the IC, such that the latter (initially conceived to allow for the passage of only gases or steam) could also become permeable to liquids, depending on the hydrostatic pressure of the wound. Furthermore, whereas the mechanism of action of CVDs has been conceived for Stage II, or the Granulation Stage, of wounds (hence another name of CVD foam: “granufoam”), the IVDs, due to the atraumatic or protective nature of their ICs, respect the wounds and make it possible to protect epithelial neo-formation, for which reason they would also be indicated in Stage III, or the Epithelialisation Stage.

But, moreover, the IVDs may be used both as primary dressings (i.e. directly on the lesion) and secondary dressings, on practically all the primary dressings available in the market; and, moreover, they may also be used in Stage I, or the Debridement Stage, of lesions (wherein CVDs would be contraindicated), since they are compatible, amongst others, with products such as hydrogels, hydrofibres and alginates; and even with enzymatic debridement ointments or creams; this makes it possible to promote different types of debridement (e.g.: surgical, enzymatic, autolytic, etc.). Thus, as an example, they may enhance hydrofibre primary dressings that absorb large quantities of exudate and bacteria from the wound, forming a soft, cohesive gel that perfectly adapts to the surface of the wound, keeping the humidity; however, given the limited absorption capacity of these primary dressings, the application of IVDs thereon (as secondary dressings), due to their hydroregulatory potential, with an absorption capacity that is unlimited (the excess exudate is driven to an external reservoir) and controlled (depending on the pressure parameters applied), will make it possible to optimise autolytic debridement.

Thus, we can verify the advantages provided by the IVD, since it combines faster placement and greater safety and comfort for the patient, improving the efficiency and effectiveness, since, in addition to being able to be applied directly on the cutaneous surface, it may be kept in place for a greater number of days than the current system of coupling dressings, since it makes it possible to distribute a uniform pressure in the area of application (without the hyper- or hypopressure risks derived from overlap or shearing between the juxtaposed/superimposed dressings), thereby providing a greater cutaneous retraction and reduction of stress on the suture lines.

Thus, whereas, due to the protective nature of their IC, the IVDs respect the wounds, the mechanism of action of CVDs has been conceived to produce stress or stimulation on the lesion bed in order to stimulate the proliferation of granulation tissue therein. However, this same stress, in addition to being capable of causing “exhaustion” in the body's response thereto, which would require suspending this type of therapy, prevents them from coming in contact with the healthy perilesional skin or other noble organs or tissues at the core of the wound. All this entails dedicating extra time, not only to cut them and adapt them to the shape of the wound, but also to prevent them from coming in contact with the noble structures present therein.

Thus, the open-pore structure of CVDs, designed to stimulate-stress the wound bed, favours the macroinvasion of said pores by the granulation tissue present in the lesion bed, such that it is not recommendable to keep them “in situ” for more than 2-3 days, due both to the risk of overinfection (since drainage of the wound is blocked, due to obstruction of the CVD pores) and, moreover, to a number of consequences that must be borne in mind: upon removing the dressing, the cures tend to be painful, since oftentimes the CVD has to be literally “torn off” the wound bed, which involves damage to the neo-formed granulation tissue; this entails a regression in the healing process. Moreover, said “tearing-off” entails a real risk of haemorrhages; for this reason, these CVDs must be used with precaution (especially in patients with coagulation alterations); moreover, another danger of the aforementioned “tearing-off” is that fragments of the CVD may be retained in the core of the lesion, with the consequent risk of infections or sepsis; in fact, a recent FDA report (Feb. 24, 2011. FDA Safety Communication: UPDATE on Serious Complications Associated with Negative Pressure Wound Therapy Systems), has warned about the potential dangers associated with this type of dressings, indicating cases of death due to the possible haemorrhages or infections linked thereto.

On the contrary, as mentioned above, the atraumatic nature of the IC of the IVD protects the system against exhaustion, since it protects the granulation tissue, preventing it from coming in contact with the open-pore structure of the OC; for this reason, it prevents blockage of the drainage of the lesion exudate, does not cause regression in the healing process of the wound during changes of this dressing, such that the interval between said changes may be extended for even more than one week, as compared to 2-3 days for CVDs, and, moreover, since, unlike CVDs, the IVD is atraumatic, it favours epithelialisation and even allows for the enhancement thereof, by making it possible to associate supplementary therapies, such as stem cells, growth factors, amongst others.

Consequently, the IVD of the present invention is different from CVDs in that it further incorporates an IC whose contact surface with the body makes it suitable to come in contact not only with the lesion bed, but also with the skin, as well as, potentially, with noble tissues or structures of the wound. This makes it possible to expand the indications of this vacuum therapy not only to promote healing in losses of substance, but also as a valuable tool in those situations that require compression, with or without loss of substance.

Moreover, the fact that it is an integrated dressing, also applicable to intact cutaneous surfaces, increases the effectiveness thereof, since it allows for the development of tangential centripetal pressures on intact cutaneous surfaces, and also directly beneath the inner surface of the core of said dressing, thereby providing a greater cutaneous retraction and reduction of stress on the suture lines of closed wounds or contraction of the bed in open wounds; i.e. also enhancing centripetal compression. All this is optimised even further when IVDs with cores with an adhesive inner surface are used. Moreover, in order to favour drainage, the IVD may further incorporate a device composed of modified internal Redon drains that will avoid the need to use external Redon drains.

Since it can also come in contact with the perilesional skin, the IVD of the invention does not need to be cut according to the shape of the wound, unlike CVDs, which, if they come in contact with the skin, produce irritation and maceration; in addition to the consequent savings in time and much faster learning curves, it has the beneficial effect that, since it may be applied beyond the lesion itself, it acts as a better safety seal for adhesion of the dressing, there is a lower risk of leakages or separation of the dressing, and it produces a greater expansion effect on the perilesional skin due to the aforementioned tangential centripetal forces. Thus, in open wounds or losses of substance, the dressing of the present invention allows for a greater contraction effect on the wound than CVDs, whereas in closed wounds stress reduction on the cutaneous sutures is enhanced.

Despite its benefits, traditional “passive” compression therapy, performed by means of conventional belts or bandages, produces a potentially dangerous compression, since it is circular, or non-selective in regards to the area to be treated; with an intensity that is hard to control, since it is not technically measurable; and not centripetal; and it may even cause distraction or separation of the edges of the wound beneath the surface of the dressing, due to the “battering ram effect”; and, moreover, it does not make it possible to integrate the system of drainages or Redon drains from the lesion bed therein.

On the contrary, the “aspirative” compression therapy of the IVD not only allows for a selective, centripetal, controlled compression as a function of the pressure parameters to be applied, but also, through its own aspiration system by means of modified redon drains, makes it possible to dispense with external Redon drains, which makes it possible to optimise the patient's safety and comfort even further, favouring precocious mobilisation and deambulation, and reducing the risks associated with prolonged immobilisation, such as pulmonary thromboembolism and others. The IVD has been proven to be a tool in those situations wherein an anti-oedema therapy may be beneficial, whether in open or closed lesions, whether post-operative or for other reasons, e.g. post-traumatic. As a result of the above, the IVD may be used simultaneously on plasties associated with grafts, contributing not only to safety, but also to a more aesthetic result, by allowing for a better homogenisation of both surfaces.

An additional advantage of IVDs is that they may have all the constitutive elements thereof (IC+OC+sealing film+other specific elements) in their commercial delivery form, since, given that they do not require to be cut according to the shape of the wound, they may, unlike CVDs, which are assembled piece by piece at the time of placement, be manufactured with all the elements in an integrated fashion; for this reason, they would not require a learning curve. Thus, it makes it possible to expand the indications of vacuum pressure therapy to very small-size appendices or anatomical areas, even those with irregular surfaces, whether fixed (e.g. nasal tip, finger and toe tips, etc.) or mobile (such as losses of substance in the finger and toe joints), which may present losses of substance with a very small diameter (sometimes less than one centimetre), albeit complex (e.g. osseous, tendinous, chondral or articular exposure). As, it would be feasible to have “mini” IVDs integrated with “mini” aspiration tubes incorporated in a manufactured manner (it would be sufficient to remove the protective flaps from the adhesive surface thereof and apply them on the surface of the body); they are advantageously interesting in the case of these complex losses of substance with a small diameter; this, in addition to facilitating a faster placement, would allow for the selective application of vacuum therapy in areas wherein it is currently still not operative.

Permeability of the IVD

The permeability of the IC of the IVDs may vary or be adapted depending on the needs; as, their permeability may be increased modified manually in situ at the time of placement simply by performing, when placed on the wound, the aforementioned perforations to pierce the IC; said perforations may be made to correspond (“like a mirror”) with the lesion areas that are potentially more exudative.

Classification of the Indications of VAT, Using the IVD

Thus, schematically, some of the indications of the two main lines of this Anti-oedema Therapy, whereto we have referred, for a better understanding of their mechanism of action, as Vacuum-Assisted Therapy (VAT), using the IVD, would be:

1. VAT to be applied on closed wounds or lesions

A. To increase the safety or comfort of post-operative treatment.

B. Prevention/Treatment of pathological scars.

C. Treatment of oedemas of non-surgical origin.

D. Stabilisation or splinting following sprains, luxations or even osseous or osteo-articular lesions.

E. As an “active” compression dressing-belt.

F. As a perilesional skin and long-distance expander.

2. VAT to be applied on open lesions or wounds
A. To increase the safety or comfort of post-operative treatment.
B. To promote healing by secondary intention, i.e. that the lesion ends up closing without the need for reconstructive surgery.
C. To perform a deferred closure or reconstruction:
   To reduce surgical requirements, i.e. that the lesions improve and, therefore, the reconstructive surgery may be less aggressive.
   Whilst awaiting histological, bacteriological, etc., confirmation.
D. Stabilisation or splinting following open osseous or articular lesions, with or without loss of substance.
E. As an “active” compression dressing-belt.
F. As a perilesional skin and long-distance expander.

### SUMMARY OF THE IVD

Thus, the IVD has a number of characteristics in the application thereof with respect to the CVDs in the state of the art, due to its design and the protective or atraumatic nature of its IC, which makes it possible for it to come in contact with healthy skin and, even, with various noble tissues or structures of the wound bed, its main characteristics being:
1) Although the IVD could be cut according to the shapes of the wounds and be applied in the same way as CVDs (even coming between the edges of said wounds), it does not require to be cut according to said shapes of said wounds. For this reason, it does not have to come between said edges of said wounds and, therefore, does not limit approximation of said edges; and also it is possible to have dressings with all their components in a manufactured manner, to be directly applied on the surface of the body, on both open and closed wounds, with a significant savings in time and increased safety.
2) As a result of the above, it is also possible to have improved integrated “mini” vacuum dressings in a manufactured manner, with “mini” aspiration tubes incorporated therein (i.e, “mini” IVD) in order to expand the indications of vacuum pressure therapy to appendices or very small-size anatomical areas, wherein currently it is still not operative.
3) Its contact with the perilesional skin or even long-distance provides significant savings in time and a stronger seal, thereby reducing the risk of leakages or peeling off.
4) It produces a greater perilesional skin expansion effect, than CVDs, due to the enhancement of tangential centripetal forces; thus, in open wounds or losses of substance, it allows for a greater contraction effect in the wound; whereas in closed wounds stress reduction at the level of the cutaneous sutures is enhanced; so, the IVDs may be used in lesions or wounds, open or closed, as a perilesional skin expander or even at a distance.
5) Due to their variable permeability, the IVDs may potentially be applied, as a function of the needs, in any of the three healing Stages of open wounds (unlike CVDs, also called “Granufoam”, which were conceived only for Stage II, or the Granulation Stage); furthermore, the atraumatic nature of its IC protects the granulation tissue and prevents contact thereof with the open-pore structure of the OC and blockage of the drainage of the lesion exudate; thus, it prevents regression of the healing process during dressing changes. As a result, the interval between said changes may reach 1-2 weeks, as compared to 2-3 days for CVDs. Another advantage of the IVDs is that they may even be used in Stage I of wound therapy, in order to favour Debridement of the wound bed, since they may be associated with both debriding enzymes and products with minimum permeability, hydrofibres, alginates and hydrogels, which, on the contrary, are incompatible with CVDs. And also, a significant advantage of the IVD is that the fact that it is atraumatic favours Stage III, or the Epithelialisation Stage, and it is even possible to enhance it by associating it with supplementary therapies, such as the use of stem cells, growth factors, amongst others.
6) Another relevant property of the IVD is that it allows for an “aspiration” compression that, amongst others, combines the properties of being selective, centripetal and controlled; and, by using modified internal redon drains, makes it possible to dispense with external redon drains. This optimises the patients' safety and comfort as compared to current belts or bandages.
7) The IVD may be used simultaneously on plasties associated with grafts, contributing both to safety and to a more aesthetic result, since it makes it possible to better homogenise both surfaces.
8) The IVD, unlike the CVDs, which come between the edges of the abdominal wound, limiting the approximation thereof, may be used most successfully in the “open abdomen” pathology. Since the IVD may be applied directly on the perilesional skin, and also at a distance, centripetal approximation forces are developed on the edges of the abdominal wound that cause a true expansion effect on the abdominal wall, which contributes to favour direct fascial closure.
9) Due to the atraumatic or protective character of its IC, in addition to being capable of coming in contact with the cutaneous surface, the IVD may come in contact with various noble organs or tissues present in the lesion bed, which include the visceral surface itself.
10) Unlike the current CVDs, the permeability of the IVDs is easily customisable “in situ” to the needs of the lesion bed.
11) The IVDs may be used in the prevention and treatment of pathological scars.
12) The IVD dressing of the invention may be kept “in situ” for a significantly greater number of days than CVDs; therefore, it may be recommendable for the sealing film that adheres to the skin, and even the IC itself, to optionally have antimicrobial impregnation.

### DESCRIPTION OF THE INVENTION

The Perfected IVD of the Present Invention and the VAT

To this end, the present invention proposes the perfecting of the aforementioned IVD; either to expand even more the indications of VAT or to increase the flexibility or versatility of application of said dressing in different types of lesions, open or closed, with or without loss of substance, wherein vacuum-assisted anti-oedema therapy may be beneficial, and which appears as a novelty within its field of application, since, according to the implementation thereof, the aforementioned objectives are satisfactorily met; the characteristic details are adequately recorded in the claims that accompany the present specification.

In principle, any open-pore element, regardless of whether it is made of foam or any other material (e.g. 3D nylon fabric, etc.), provided that it is susceptible to retracting by applying vacuum, and has an inner area or an inner surface capable of protecting the surface of the body, both in open or closed wounds or lesions, against the direct action of the vacuum, could form the core of a Perfected IVD.

Permeability of the Perfected IVD

The permeability of the IC of the IVDs may vary or be pre-established in a manufactured manner:

a) in order to increase the permeability thereof:
IC with “manufactured” perforations (e.g. linear, to be applied on a sutured wound; central, like a sheet, for application on a loss of substance).
modifications at the level of the layers thereof, e.g. by enhancing or optimising, in a manufactured manner, the hydrophilic character of the limiting layer of the IC; in order to have Perfected IVDs with said ICs of a predictable and “self-regulatory” permeability which, in the absence of aspiration, were not permeable to liquids, but, when subjected to the aspiration source, on a wound with a significant exudative potential, would become permeable to liquids, without the need of added perforations.

b) in order to reduce the permeability thereof:
modifications at the level of the outer or inner layers thereof; e.g. transforming them, in a manufactured manner, such that they have minimum permeability (which, in turn, could be manually modified “in situ”, by performing punctures or perforations, depending on the needs).

Advantages of the Perfected IVD

So, an important consideration in regards to the Perfected IVD of the present invention is that it is possible to have semi-occlusive or minimum-permeability ICs, for application even on orifice or juxtaorifice areas, such as intestinal fistulae, perianal areas, juxta-tracheostomies, etc., without the risk of aspiration of intestinal liquid or air leakages, since the vacuum is established solely in the interior of the dressing, between its outer sealing film and its IC, and is not transmitted to the space between the dressing and the surface of the body.

Similarly to the IVDs (and unlike CVDs), the Perfected IVD may have also all the constitutive elements thereof (IC+OC+sealing film+other specific elements) in their commercial delivery form, since, given that they do not require to be cut according to the shape of the wound, they may, unlike CVDs, which are assembled piece by piece at the time of placement, be manufactured with all the elements in an integrated fashion; for this reason, as the IVDs, they would not require a learning curve.

The shape of the Perfected IVD may adopt various configurations, such as flat, curved, or other specific forms depending on the shape of the surfaces of contact with the body; for example, a semi-cylindrical configuration for better adaptation to surfaces with a semi-cylindrical shape, such as legs, arms, thighs, etc.; a concave configuration for better adaptation to the cranial vault, heel, concave-shaped articular surfaces, such as the knee, elbow, etc.; a saddle-shaped configuration for better adaptation to surfaces such as the neck; and, finally, other adequate, more complex configurations for different body sites, which will be known to persons skilled in the art. And even, can be manufactured Perfected IVDs with diameters even smaller than 1 cm, which could even have a concave shape or other geometrical configurations for better adaptation to the surface of application.

It would also be possible for the integrated core, composed of the junction of the OC and the IC, to be manufactured independently from the rest of the IVD components.

Also, it would be possible for the junction or integration between the OC and the IC to be reversible, either because the contact surfaces between them have been endowed with a more labile adhesive in one or both directions, allowing for repeated removals and placements, or because it is a reversible adhesive junction of the type protected by reversible adhesive flaps, or even because it is a “velcro”-type junction.

The IVD can also be manufactured to be Perfected with complete oblique cuts (in one or more directions) at the OC level, since, given that the latter is bound to the IC, it would act as a bridge, such that the segments of the OC do not separate; this would make it possible to adapt said Perfected IVD to curved or even convex surfaces.

Similarly, in order to favour a greater versatility of the IVD in its adaptation to more irregular surfaces (e.g. concave-convex, etc.), it would be useful to Perfect said dressing with oblique cuts appearing both on the outer face and the inner face of the OC. In this case, the oblique cuts should be subtotal, in order to prevent the OC from separating into several pieces. However, it would also be possible for the OC to be completely cut following a total oblique spiral cutting pattern, which would confer it with the property of adapting to both concave and convex surfaces; it would be sufficient to turn it around in one direction or the other.

### DESCRIPTION OF THE DRAWINGS

In order to supplement the present description and contribute to a better understanding of the characteristics of the invention, the present specification is accompanied by, as an integral part thereof, some figures, wherein, for illustrative, non-limiting purposes, the following has been represented:

FIG. 1 shows a schematic, disproportionately enlarged representation, designed to facilitate understanding, of a sectional view of the IVD of the PCT2010000221, applied, in this case, on a closed surgical wound as a vacuum-assisted therapy dressing, where one may observe the main parts and elements that it comprises, as well as the configuration and arrangement thereof.

FIG. 2 shows a schematic view of said IVD of FIG. 1, also on a closed surgical wound, wherein a drainage device has been incorporated into the IVD.

FIG. 3 shows a schematic view of said IVD, applied to a case of a complex open wound, with loss of substance and dehiscent edges, wherein a drainage device has been incorporated into the IVD.

FIG. 4 shows a schematic view of a Perfected IVD of the present invention located on the knee of a patient.

FIG. 5 shows a schematic view of a Perfected IVD of the present invention located on the neck of a patient.

FIG. 6 shows a schematic view of a Perfected IVD of the present invention located on the limb of a patient.

FIGS. 7 and 8 show two Perfected IVDs for the open abdomen that incorporate a second inner component in an integrated or pre-integrated manner.

### SOME EXAMPLES OF PREFERRED EMBODIMENTS OF THE IVD WITH SOME OF ITS POSSIBLE WAYS OF PERFECTING

In view of the aforementioned figures, and in accordance with the numbering adopted, therein we may observe preferred embodiment examples of the invention, which, without being limited thereto, comprise the constitutive parts and elements indicated and described in detail below.

### Embodiment No 1

### Components

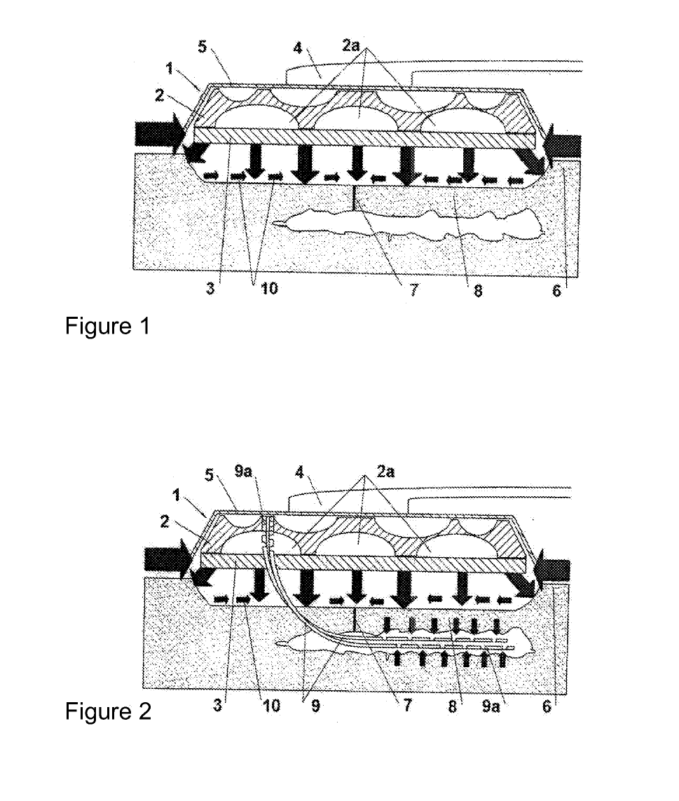

FIG. 1 shows a general configuration for application of the mentioned IVD on a closed surgical wound. Thus, as may be observed in said figures, dressing 1 in question is sealed by means of adhesive film 5 and said dressing is adhered to surrounding skin 7 of the surgical wound, and may even exceed skin 6, which overlies the area of surgical detachment 8; an aspiration tube 4 connected to a vacuum pump, which is not represented, is coupled to said adhesive film.

Beneath its sealing film, this IVD is essentially composed of two areas, 2 and 3:
an outer component 2, with open pores 2a.
and an inner component 3, with variable permeability, which may or may not be adhesive.

Both in this FIG. 1, and in the next one, FIG. 2, the following have been marked with arrows: the positive or sagittal compression pressures exerted by outer component 2 and inner component 3 of dressing 1, the tangential centripetal pressures exerted by outer adhesive film 5, which adheres to skin 6 in the periphery of the dressing; as well as the tangential centripetal pressures developed beneath adhesive surface 10 of the dressing.

### Embodiment No. 2

### Drainage of Closed Wounds

The use of IVDs, as compared to traditional bandages and dressings, makes it possible to optimise the reduction of post-operative oedemas, favouring precocious mobilisation and reducing the risk of dehiscences of the surgical wound (i.e. that it re-opens due to precocious mobilisation). Moreover, the system of modified Redon drains 9a of FIG. 2 makes it possible to eliminate the risk of infection associated with the use of traditional external Redon drains.

Thus, FIG. 2 shows the dressing of FIG. 1, wherein one may observe outer component 2, which has the peculiarity of having a certain thickness that may differ depending on the type of lesion and the area of the surface to be treated; this favours a more effective anti-oedema therapy, and makes it possible to incorporate a drainage device, which, as may be observed in the Figure, is composed of redon drains or tubes 9 that present, in a manufactured manner, a plurality of orifices 9a at the two ends thereof; of these ends, one is inserted in the outer open-pore area or component 2, and the opposite end is designed to be inserted in surgical plane 8, such that said tubes 9 directly transmit the reduced pressures from said plane to the outer open-pore component 2, thereby promoting tissue adhesion.

Moreover, the arrows in FIG. 2 show the reduced pressures exerted by orifices 9a at the end of the redon drains 9 inserted in underlying surgical plane 8. The arrows also indicate the positive or sagittal compression pressures exerted by outer component 2 and inner component 3 of dressing 1, the tangential centripetal pressures exerted by outer adhesive film 5, which adheres to the skin in periphery 6 of the dressing; as well as the central tangential pressures developed beneath adhesive surface 10 of the dressing.

### Embodiment No. 3

### Drainage of Open Wounds

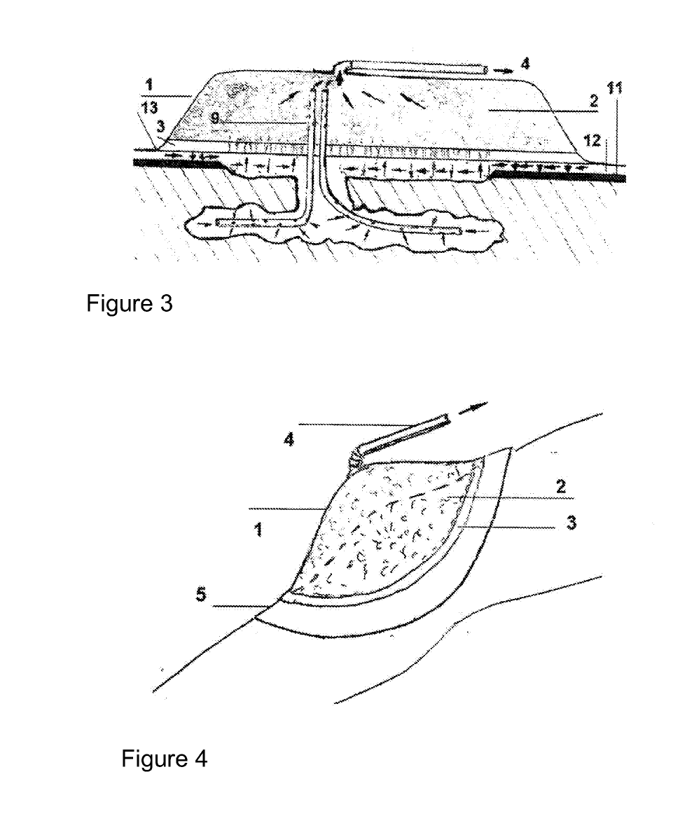

FIG. 3 shows a schematic view of the IVD of FIGS. 1 and 2, applied to a case of a complex open wound, with loss of substance and dehiscent edges, wherein a drainage device has also been incorporated into the IVD, which consists of redon drains or tubes 9 that present, in a manufactured manner, as in FIG. 2, a plurality of orifices at the two ends thereof; of these ends, one is inserted in the outer open-pore area or component 2, and the opposite end is designed to be inserted in surgical plane 8, such that said tubes 9 directly transmit the reduced pressures from said plane to outer component 2. Moreover, one may observe skin 11, “virtual” space 12, excessively enlarged for clarity, between skin 11 and IC 3, and area 13, where it joins adhesive or non-adhesive inner surface 10 of dressing 1 with adhesive sealing film 5.

In FIG. 1, as well as in FIGS. 2 and 3, the following have been marked with arrows: the positive or sagittal compression pressures exerted by outer component 2 and inner component 3 of dressing 1, the tangential centripetal pressures exerted by outer adhesive film 5, which adheres to the skin on the periphery of the dressing; as well as the tangential centripetal pressures developed beneath surface 10 of the dressing; moreover, in FIG. 3, since it is an open wound with loss of substance, the reduced sagittal pressures on the lesion bed have also been marked with upward arrows.

### Embodiment No. 4

### Application on Plasties (Flaps)

The main object of the application of traditional compression therapy on plasties is to obtain an anti-oedema effect that favours vascularisation; however, since the compression does not have a controlled intensity and is not centripetal, it is possible for excessive compression to have the opposite effect and cause vascular compromise, as well as distraction in the suture lines (which may cause dehiscences or necrosis).

On the contrary, as already mentioned, VAT, using the IVD of the PCT2010000221, has the advantage that it provides compression with a controlled intensity and relaxation of the suture lines and, moreover, unlike traditional compression, it is selective, which contributes to increase the safety of the reconstruction.

One example would be the application of post-operative compression therapy in “tummy-tucking” surgery, also called abdominal plasty or abdominoplasty.

This is one of the surgical procedures with the greatest risk of pulmonary thrombosis or embolism, and the best post-operative prevention would consist of allowing the patient's precocious deambulation. However, the limited reliability of the traditional post-operative treatment, which, by means of conventional circular compression belts or bandages, produces a low-quality, non-selective, uncontrolled, non-centripetal compression, may cause post-operative deambulation or mobilisation to be delayed. Moreover, in addition to covering the operated area, this type of compression also includes the back and extends to the proximal part of the thighs, where it also exerts a circular compression that could predispose to thrombosis in the lower limbs; this would exponentially increase the risk of pulmonary embolism. Furthermore, this type of traditional compression would not avoid dispensing with the use of external Redon drains.

On the contrary, the IVD would simultaneously be: a dressing and an “aspiration” compression belt that, in addition to providing a selective, controlled, centripetal compression, makes it possible to incorporate a system of Redon drains integrated therein. Consequently, it makes it possible not only to dispense with conventional bandages and external redon drains, but, by increasing the safety of the reconstruction, reduces the risk of haematomas or of the abdominal flap coming off, which, in turn, allows for precocious post-operative deambulation; this, in addition to the absence of circular compression on the thighs, will minimise the risk of pulmonary thromboembolism (PTE) even further.

Vacuum dressing 1 used is the same type of dressing shown in embodiments 1 and 2 and FIGS. 1 and 2, with the corresponding constitutive elements thereof; the OC of said dressing could have a much greater thickness than that of current CVDs, e.g. about 6 cm, as compared to the 3-cm thickness of CVDs; moreover, given its large surface of application, said OC could incorporate drainage devices with elements designed for adaptation to retraction of the dressing and said drainage devices could even have a spiroidal shape in order to optimise said adaptation to contraction of the dressing even further.

Furthermore, for a greater flexibility or versatility of application of the IVD of the PCT2010000221, said dressing, as already mentioned, can be Perfected in the present invention in several ways (e.g: possibility to be manufactured with oblique cuts in several directions, . . . )

### Embodiment No. 5

### Application on Plasties Associated with Grafts

Traditionally, because its intensity is not controlled, in the case of a plasty associated with a graft, conventional compression therapy is performed independently for each of them. For this reason, sealing of the point of junction between them is the Achilles' heel of reconstruction. Its relevance is oftentimes only aesthetic, but sometimes, as in the case of tracheal or neurosurgical reconstruction, amongst others, it may entail failure of the reconstruction because it becomes a source of leakages, of saliva or cephalorachidian fluid, respectively, and may even put the patient's life at risk.

The application of a single dressing on the entire area of both allows not only for a stronger sealing at the point of junction between the plasty and the graft, with a more homogeneous and aesthetic surface, but also, very importantly, a more controlled, safer compression.

Besides, for a greater flexibility or versatility of application of the IVD, said dressing, as already mentioned, can be Perfected in several ways (e.g: possibility to be manufactured with oblique cuts in several directions, . . . ). Moreover, in the case of neurosurgical reconstruction, it is very useful to have Perfected IVD dressings with a concave inner surface, for better adaptation to the convexity of the cranial vault.

### Embodiment No. 6

### Knee Reconstruction

FIG. 4 shows a schematic view where we may observe, externally, a Perfected IVD 1 with outer component 2, inner component 3, aspiration tube 4 and adhesive film 5 of embodiment No. 1 or No. 2, located on the knee of a patient. So, in the case of prosthetic knee reconstruction, the IVD can be Perfected with a concave inner contact surface thereof, for better adaptation to the convex morphology of the knee. Moreover, it would allow for a selective dynamic stabilisation or splinting, and may incorporate lateral, upper and lower reinforcement extensions or tabs, in order to reinforce the adhesion thereof during flexo-extensive movements of the knee, which could give it a “butterfly wing” configuration. Though, said dressing, as already mentioned, could also have been Perfected, in other ways (e.g: possibility to be manufactured with oblique cuts in several directions, . . . ).

### Embodiment No. 7

### Corrective Surgery of Dorso-Lumbar Scoliosis

Another example of the use of the IVD as a “vacuum-assisted dressing-belt” according to embodiments No. 1 and No. 2 is the post-operative use thereof following corrective surgery of dorso-lumbar scoliosis in order to act as a controlled-compression “dressing-belt”.

The OC of said dressing could have a much greater thickness than that of current CVDs (e.g. about 6 cm, as compared to the 3-cm thickness of CVDs); likewise, given its large surface of application, said OC could incorporate drainage devices similar to those described in embodiment 4.

The IVD, as already mentioned, could also have been Perfected to increase the flexibility or versatility of application of said dressing, in several ways (e.g: possibility to be manufactured with oblique cuts in different directions, . . . ).

### Embodiment No. 8

### Post-Operative Handling of Neck Surgery

The current post-operative compression method consists of a circular compression bandage on the neck which causes discomfort to the patient and even facial oedema or respiratory difficulty; moreover, it requires the use of external redon drains and the corresponding bottles.

However, with the IVD of the PCT2010000221, it would be sufficient to apply a dressing according to embodiments Nos. 1 or 2, endowed with a semi-permeable IC 2, which could incorporate modified redon drains 9a, see FIG. 2, in the interior thereof.

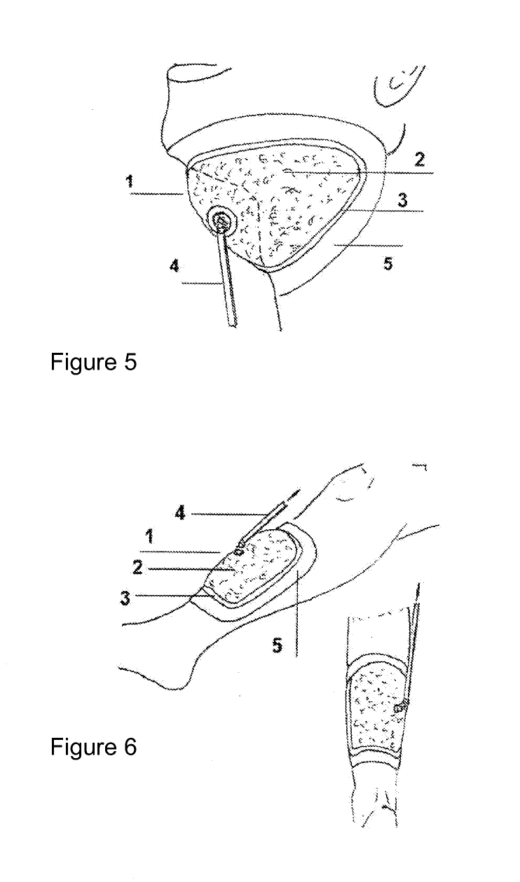

Moreover, for better adaptation to the morphology of the anterior region of the neck and the lower region of the jaw, which is reminiscent of a saddle, it would be recommendable to use a Perfected IVD with its inner face adopting a “saddle” shape. FIG. 5 shows a schematic view of said Perfected dressing 1 of the invention, where we may observe outer component 2 and inner component 3, as well as aspiration tube 4 and sealing film 5, located on the neck of a patient.

### Embodiment No. 9

### Post-Lipoaspiration Compression

The IVD of the PCT2010000221 may also be used to act like a real “vacuum-assisted compression belt”. This is especially useful in those areas subjected to lipoaspiration, wherein conventional belts are difficult to apply due to their non-selective action.

The OC of said dressing could have a much greater thickness than that of current CVDs (e.g. about 6 cm, as compared to the 3-cm thickness of CVDs); likewise, when applied on a large surface of the body, said OC could incorporate drainage elements or devices of the type mentioned in embodiment 4.

Said IVD could also have been Perfected to increase the flexibility or versatility of application of it, in several ways (e.g: possibility to be manufactured with oblique cuts in different directions, . . . ).

### Embodiment No. 10

### Prevention and Treatment of Pathological Scars

A pathological hypertrophic or keloid scar is understood to be a scar that remains raised, red and widened for months or even years following the surgical procedure. It is worth noting that there is no wound, since the skin is intact.

Currently, the most effective conservative treatment consists of microporous adhesive films made of polyurethane, e.g. Trofolastin®, manufactured by Novartis; or of silicone, Mepiform®, manufactured by Molnlycke Health Care. The dressing provides a certain degree of compression, which may be enhanced in the limbs by means of circular bandages or tubular belts; whereas in other areas, such as the pre-sternal area, due to the discomfort or difficulty to establish this type of compression by means of belts or bandages, manual compression of the films is recommended, at least periodically, in order to enhance the effect thereof. However, an adverse effect of this type of compression is the battering-ram effect, already mentioned, which causes a greater stress-distraction of the scar.

On the contrary, since it develops a centripetal compression, the IVD of embodiment 1 will simultaneously allow for the reduction of tangential stress in the scar area. The innermost layer is adhesive, made of silicone or polyurethane, although it may also be of any other compound in polymer technology that is useful for this type of pathologies.

For a greater flexibility or versatility of application of said IVD, as already mentioned, it can be Perfected in several ways (e.g: possibility to be manufactured with oblique cuts in several directions, . . . ).

### Embodiment No. 11

### Application on Skin Graft Donour Sites

In patients in whom coagulation cannot be suspended, either due to an urgent procedure or because it is contraindicated, obtaining a graft entails a marked tendency to bleed at the donour site.

FIG. 6 shows an example of an IVD which has been Perfected with a hemicylindrical configuration, applied on the anterior and posterior curved regions of the leg.

Unlike circular compression bandages, which are contraindicated in this type of patients due to the risk of thrombosis, this dressing makes it possible to exert a more effective, selective, non-circular compression. One of the most frequent donour sites is the anterior face of the thigh; given the curved shape of the thigh, the dressing could also be Perfected with a hemicylindrical configuration.

### Embodiment No. 12

### Decubitus Ulcers

The etiology of decubitus ulcers is positive pressure. For this reason, use of the CVD of the prior state of the art may even aggravate the lesion, since, as already described, the final pressure resulting from Reduced Pressure Therapy is Positive.

However, since it may applied on the intact cutaneous surface, even at a distance from the edges of the lesion, the IVD dressing of embodiment No. 3 (with or without incorporated Redon drains) makes it possible for the compression generated by the dressing to be distributed over a larger surface area, and the positive pressures on the depressed ulcer bed are minimised and enhances perilesional sealing, with the corresponding reduction of the risk of leakages.

For a greater flexibility or versatility of application of the dressing, as already mentioned, it can be Perfected in several ways (e.g: possibility to be manufactured with oblique cuts in several directions, . . . ).

### Embodiment No. 13

### Digestive Fistulae

Intestinal fistulae is a pathology that is traditionally refractory to vacuum pressure therapy. This is essentially due to the fact that, with the currently available systems, there is a risk of uncontrolled aspiration of intestinal secretions, and even of repermeabilisation of other fistulae that might be debitless or silent; this risk is due to a paradoxical effect: if we increase the aspiration pressure in order to enhance the contraction effect of the dressing so as to favour the adhesion thereof to the surface of the body, thereby contributing to sealing said fistulae, the latter could be aggravated, or an awakening or reactivation of other fistulae that might be silent could be enhanced, even causing risk of death to the patient. On the contrary, if we reduce the aspiration pressure in order to decrease the fistula debit, we will also reduce the contraction effect of the dressing, which will increase the risk that the vacuum dressing will come off and, moreover, will reduce the compression sealing effect on the fistula(e).

In order to solve this problem, current practise consists of individually isolating each of these fistulae in order to direct them to other external drainage systems. However, this is an extremely laborious and tedious process, with numerous connections that reduce the patient's comfort and increase the risk not only of leakages, but, moreover, that the fistulae perpetuate themselves.

Thus, amongst the possible forms of application thereof, and as a non-limiting example, an IVD dressing of embodiment No. 1 could be used. Furthermore, to avoid the risks of leakage, a Perfected IVD with a minimum-permeability, adhesive IC 2, that may be even similar to adhesive sealing film 5 that also covers the dressing; whereas a primary dressing, preferably with anti-microbial activity, could be applied on the bleeding perifistular surface; the dressing could be limited or, on the contrary, protrude, like a wick, from the lower part of said dressing. Thus, this system would make it possible to either isolate the fistula(e) by means of sealing or, on the contrary, channel the debit thereof, beneath the Perfected IVD, through the “wick” of the primary dressing; therefore, regardless of the no. of fistulae, and without the need to individually isolate them, said debit, for example, could even be driven towards a colostomy bag. This would increase the compression component on the fistula(e) and approximation of the edges of the wound, in order to promote the sealing of said fistula(e), without the risk of uncontrolled aspiration of intestinal secretions, or of repermeabilisation of other fistulae that could be debitless or silent.

And also, said Perfected IVD, to increase the flexibility or versatility of application of it, can also be perfected even more (e.g: possibility to be manufactured with oblique cuts in different directions, . . . ).

### Embodiment No. 14

### Treatment of Patched Losses of Substance

A relevant application of the IVD of the PCT2010000221 is the treatment of a lesion area with multiple concomitant wounds, alternating with areas of intact skin.

The use of CVDs would entail cutting a different dressing for each lesion and the further need to establish connection bridges between them in order to transmit the vacuum to the aspiration source, with the consequent loss of time and increased duration of the operation.

On the contrary, by using said IVD, it will be sufficient to apply a single dressing on the entire lesion area and pierce the IC, for example, with a needle or a scalpel blade in the areas corresponding to the loss of substance where aspiration of the exudate is to be most enhanced; or optionally, a Perfected IVD wherein said inner component (3) of said vacuum dressing (1), has a predictable and “self-regulatory” permeability which is not permeable to liquids in the absence of aspiration, but, if applied on exudatives areas, will become permeable to liquids when subjected to an aspiration source, without the need of added perforations.

Besides, said dressing can also be Perfected, as already mentioned, with manufactured oblique cuts in different directions, etc.

### Embodiment No. 15

### Prevention and Treatment of Dehiscent Wounds

Dehiscent wounds consist of pathological openings of the wound, primarily produced following precocious removal of the stitches, in the case of wound closures performed under too much stress or in the case of inflammation/oedema, with or without an underlying infection.

Since they cannot come in contact with healthy skin, CVDs cannot be used in the prevention of these lesions. As regards the treatment thereof, they are not very effective, since, because they come between the edges of the wound, they prevent the approximation thereof.

On the contrary, the IVD of the PCT2010000221 is applied on the edges of the lesion and at a distance; this allows for a greater expansion of the perilesional skin, which facilitates closure of the wound. For this reason, the IVD is useful for both the treatment and prevention of this type of lesions.

For a greater flexibility or versatility of application of said dressing, as already mentioned, it can be Perfected in several ways (e.g: possibility to be manufactured with oblique cuts in several directions, . . . ).

### Embodiment No. 16

### Deferred Reconstruction Following Tumour Resection

Delaying the reconstruction of the defect created after extirpating a tumour has a two-fold advantage: on the one hand, it makes it possible to await the result of the histological report in order to assess whether or not it is necessary to expand the resection and, on the other hand, to improve the conditions of the wound such that the reconstruction is more conservative or may even close by secondary intention.

A non-limiting example is the use of the IVD of embodiment No. 3, with or without the incorporation of redon drains, following the resection of a tumour in the sole of the foot. The IVD not only makes it possible to increase the interval between cures, as compared to the current CVDs in the prior state of the art, but, since it provides a more effective sealing, will allow for stepping or deambulation with a lower risk of leakages.

As already mentioned, said IVD can be Perfected to increase the flexibility or versatility of its application, in several ways (e.g: possibility to be manufactured with oblique cuts in different directions, . . . ).

### Embodiment No. 17

### Open Abdomen Treatment

As already said, a relevant property of the IVDs of the PCT2010000221, unlike CVDs, is that they may also be applied directly on intact skin. A much indicated application is for the open abdomen; in fact, vacuum pressure therapy is the “gold standard” in the treatment of open-abdomen pathology. However, the use of the CVDs limits approximation of the edges of the abdominal wall, since the CVD comes between them. Thus, in order to prevent the CVD from coming between the edges of the wound, the applicant's IVD would not come between the edges of the abdominal wound, but would be applied thereon and also at a distance. Moreover, in the case that the inner surface thereof is adhesive (recommended), the centripetal approximation forces of the edges of the abdominal wound will be enhanced even further, thereby allowing for a true expansion effect on the abdominal wall that will contribute to primary/direct fascial closure

The Mild Open Abdomen, or open abdomen with minimum visceral exposure, could, in general, be treated in a similar manner to that considered in embodiment 15. Thus, the dressing would have an adhesive inner surface 10, to enhance the centripetal approximation forces of the edges of the abdominal wound even further, thereby allowing for a true expansion effect on the abdominal wall, perilesional and at a distance, which contributes to primary, direct fascial closure.

Moreover, unlike the CVD, the IVD could potentially come in contact with the visceral surface, since the latter would be protected by the atraumatic character of the IC.

For a greater flexibility or versatility of application of the IVD, said dressing, as already mentioned, can be Perfected in several ways (e.g: possibility to be manufactured with oblique cuts in several directions, . . . )

### Embodiment No. 18

### Application of the Perfected IVD With Two Inner Components

There are frequent cases of use of the Perfected IVD of the present invention where it is necessary to use two ICs, the deep IC generally being used to seal or be introduced inside a cavity area. A characteristic preferred example is the treatment of moderate or severe open abdomen. The present patent proposes a system for the treatment of the open abdomen, wherein the IVD has been Perfected by incorporating a second inner component in an integrated or integrateable manner, at least in the central area thereof; in this way, a Perfected IVD would be formed that would consist of at least two inner components, one superficial and one deep. These inner components would come in contact at their central area and, whilst the superficial inner component would be applied on the periphery, on the outer face of the abdominal wall, the deep inner component would be applied between the inner face of the abdominal wall and the visceral surface. Both inner components would be, as already mentioned, at least in contact at their central area, which could be the area of the superficial inner component with the greatest permeability; as regards the deep inner component, it will have a greater permeability both at the centre and the periphery, in order to allow for drainage even from the retroperitoneal areas.

Moreover, in order to optimise the management of the drainage of the abdominal exudate even further, both the OC of the Perfected IVD core and the 2^{nd} IC of said Perfected IVD could further incorporate their own drainage elements or devices.

Another option would have been to apply the central area of the IVD on the corresponding central area of the so-called “visceral protective layer” in the systems currently commercialised, such as the well-known ABThera® system, from KCI; though, it would also be possible to improve the drainage of said “visceral protective layer” by incorporating drainage elements or devices along its star-shaped open-pore structure core, which would make it possible to transmit the aspiration pressure to the most peripheral areas (retroperitoneal) in a more effective manner.

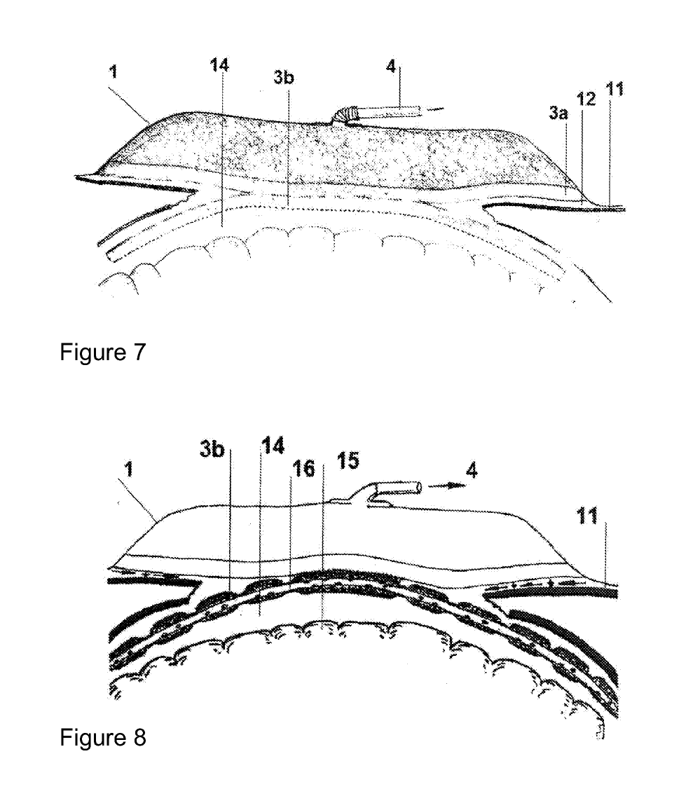

FIG. 7 shows an application example for an Open Abdomen case, which shows how, unlike CVDs, the Perfected IVD dressing 1 of the invention does not come between the edges of the abdominal wound, but is applied on said edges and also at a distance. Moreover, the Perfected IVD incorporates a 2nd IC 3b in an integrated, pre-integrated or integrateable manner, such that the two inner components, 3a and 3b, of dressing 1 of the present invention come in contact at the central area thereof, which would coincide with the area of maximum permeability of superficial IC 3a, in order to make it possible to channel the drainage from deep IC 3b, which would also be permeable in the periphery, in order to allow for drainage even from the retroperitoneal areas. One may observe space 14 between deep IC 3b and the viscerae, as well as skin 11, “virtual” space 12 between skin 11 and superficial IC 3a of dressing 1.

FIG. 8 shows a second application example to another Open Abdomen case; one may observe skin 11, viscerae 15 and dressing 1, the 2nd IC 3b whereof further incorporates drainage elements or devices 16 in the interior, thereby enhancing the drainage from the retroperitoneal areas even further than the system shown in FIG. 7.

And to increase even more the flexibility or versatility of application of said Perfected dressing, amongst other possibilities, it can also be manufactured with a concave geometrical configuration to better adaptation on the convex abdominal surface.
The Perfected IVD is a tool conceived to optimise VAT, either to expand even more the indications of the IVD of the PCT2010000221VAT or to increase the flexibility or versatility of application of said dressing in different types of lesions, open or closed, with or without loss of substance, wherein such anti-oedema therapy may be beneficial.
The IVD of the PCT2010000221 can be Perfected being manufactured in a manner, such that its IC may have minimum permeability; so, it is possible to apply said Perfected IVD even on juxtaorifice areas (e.g. perianal, juxtatracheostomies and others) or orifice areas, such as digestive, thoracic, cephalorachidian fistulae and others, without the risk of aspiration of intestinal, cephalorachidian and other liquids, and without the risk of entry of air or loss of vacuum; moreover, it is even possible to establish “wicks” beneath the surface thereof, since the vacuum is established solely inside the dressing, between its outer sealing film and its IC, without being transmitted to the space between the surface of the body and the vacuum dressing.
The IVD can also be Perfected to increase the flexibility or versatility of its application in several ways (e.g: possibility to be manufactured with oblique cuts in several directions to “universal” adaptation to different locations; possibility of being manufactured with different geometrical configurations specific for the shapes of the surfaces of contact, possibility that the union of its inner and outer components could be reversible for added versatility, etc).
For a greater flexibility of application of the IVD, it could also be Perfected by incorporating a 2^{nd} IC and, moreover, even have its own drainage device.
The Perfected IVDs may be used in lesions or wounds, open or closed, as a perilesional skin expander or even at a distance, even periorificial or yuxta-wounds.

Thus, the Perfected IVD of the present invention designed for Vacuum-Assisted Therapy (VAT) represents a new, innovative structure with structural and constitutive characteristics thus far unknown for this purpose; this vacuum dressing undoubtedly has a practical utility that will surely have a positive influence on the subsequent development of surgical techniques.

Having sufficiently described the nature of the present invention, as well as how to put it in practise, it is not deemed necessary to extend the explanation further, since its scope and the advantages arising therefrom are evident to persons skilled in the art; finally, we declare that, within the essentiality thereof, it may be put into practise in other embodiment forms with different materials and details than those indicated as non-limiting examples, and which will also have the protection that is requested, provided that the main principle, described in the following claims, is not altered, changed or modified.

What is claimed is:

1. The Improved vacuum dressing for Vacuum-Assisted Therapy of the PCT2010000221, designed for anti-oedema therapy for lesions or wounds, open or closed, which comprises an adhesive sealing film whereto an aspiration tube is attached, connected to a vacuum pump, a core located beneath said adhesive sealing film, that consists of an outer area or component, with an open-pore structure that allows for distribution of the vacuum in the interior of said vacuum dressing, and a protective or “atraumatic” inner area or component, with a variable permeability, with a surface that may or may not be adhesive, which optimise said anti-oedema therapy by regulating the vacuum; said aspiration tube determines the tangential centripetal pressures, on the cutaneous and the lesion surface, not only in the periphery of said dressing, but also beneath the surface of said dressing; wherein said vacuum dressing presents sufficient thickness to favour compression and allow for the incorporation of a drainage device; and characterized in that said improved vacuum dressing has been perfected by enhancing the flexibility or versatility of its application and even by increasing or reducing in a manufactured way the variable permeability of said improved vacuum dressing depending on the designation thereof.

2. The improved vacuum dressing that has been Perfected for Vacuum-Assisted Therapy according to claim No. 1, wherein said dressing already has all of its components in a manufactured manner.

3. The improved vacuum dressing that has been Perfected for Vacuum-Assisted Therapy according to claim No. 1, wherein, of said dressing, at least said core composed of the outer component and the inner component is presented in a manufactured manner, and one or more of said other components of said vacuum dressing may be absent in the manufacturing.

4. The improved vacuum dressing that has been Perfected for Vacuum-Assisted Therapy according to claim No. 1, wherein, of said dressing, said core composed of the outer component and the inner component is presented in a manufactured manner, and one or more of the rest of said other components according to claim 1 may be absent in the manufacturing; the junction between said outer component and said inner component is irreversible; either because the core is monoblock manufactured from the same material, with said outer component and inner component having a different morphology; or because the junction/adhesion between the different materials constituting said outer component and inner component is very strong.

5. The improved vacuum dressing that has been Perfected for Vacuum-Assisted Therapy according to claim No. 1, wherein said dressing is presented, in a manufactured manner, with said core composed of the outer component and the inner component, and one or more of said other components of said vacuum dressing according to claim 1 may be absent in the manufacturing; and because the junction or integration between said outer component and inner component is reversible, either because the contact surfaces between them have been endowed with a more labile adhesive in one or both directions, allowing for repeated removals and placements, or because it is a reversible adhesive junction of the type protected by reversible adhesive flaps or even of the “velcro” type.

6. The improved vacuum dressing that has been Perfected for Vacuum-Assisted Therapy according to claim No. 1, wherein said dressing may present various geometrical configurations specific for the shapes of the surfaces of contact with the body around the wounds.

7. The improved vacuum dressing that has been Perfected for Vacuum-Assisted Therapy according to claim No. 6, wherein said dressing presents a semi-cylindrical configuration for adaptation to surfaces with a semi-cylindrical shape, such as the legs and the arms.

8. The improved vacuum dressing that has been Perfected for Vacuum-Assisted Therapy according to claim No. 6, wherein said dressing presents a concave configuration for adaptation to concave-shaped articular surfaces such as the knee and the elbow, or even finger joints in the case of mini Perfected improved vacuum dressings.

9. The improved vacuum dressing that has been Perfected for Vacuum-Assisted Therapy according to claim No. 6, wherein said dressing (1) presents a saddle configuration for better adaptation to surfaces with the shape of a saddle, such as the neck.

10. The improved vacuum dressing that has been Perfected for Vacuum-Assisted Therapy according to claim No. 6, wherein said dressing presents a customised configuration for adaptation to even more complex surfaces of different body sites.

11. The improved vacuum dressing that has been Perfected for Vacuum-Assisted Therapy according to claim No., wherein the junction or integration between the outer component and the inner component may be reversible or irreversible, and said outer component presents, on the outer face thereof, complete oblique cuts and, since it is bound to said inner component, said inner component will act as a support so that the pieces of said outer component do not separate, and said cuts appear in one or more directions, in order to adapt said vacuum dressing in situ to complex curved or convex surfaces, and said inner component presents a surface area equal to or greater than that of said outer component, in order to prevent contact of said outer component with the skin.

12. The improved vacuum dressing that has been Perfected for Vacuum-Assisted Therapy according to claim No. 5, wherein said outer component presents, on both faces, subtotal oblique cuts in several directions, or is cut following a spiral pattern, in order to allow for adaptation of said dressing to curved, concave, convex or irregular surfaces; said inner component presents a surface area that is equal to or greater than that of said outer component, in order to prevent contact of said outer component with the skin.

13. The improved vacuum dressing that has been Perfected for Vacuum-Assisted Therapy according to claim No. 1, wherein the permeability of said inner component of said vacuum dressing is minimal, without the risk of leakages, and wherein the vacuum is established solely in the interior of said vacuum dressing, between the adhesive sealing film thereof and said inner component with minimum permeability.

14. The improved vacuum dressing that has been Perfected for Vacuum-Assisted Therapy, according to claim 1, characterised in that it comprises a second, deeper inner component, manufactured in an integrated or integrateable manner with said vacuum dressing, such that the two inner components of said vacuum dressing come in contact at their central area.

15. The improved vacuum dressing that has been Perfected for Vacuum-Assisted Therapy according to claim 14, wherein said second deeper inner component further incorporates drainage elements or devices in the interior thereof.

16. The improved vacuum dressing that has been perfected for Vacuum-Assisted Therapy according to claim No. 15, wherein drainage elements or devices have been incorporated to the Visceral Protective Layer along its star-shaped open-pore structure core, in order to transmit the vacuum to the most peripheral areas, even the retroperitoneal areas.

17. The improved vacuum dressing that has been perfected for Vacuum-Assisted Therapy according to claim No. 1 wherein modifications have been made at the level of the layers thereof by increasing, in a manufactured manner, the hydrophilic character of the limiting layer of said inner component, to have a self-regulatory permeability.
